(19) United States
(12) Patent Application Publication
(10) Pub. No.: US 2003/0165527 A1
(43) Pub. Date: Sep. 4, 2003

(54) Novel fibronectin-binding protein

(76) Inventors: Bengt Guss, Uppsala (SE); Karin Jacobsson, Uppsala (SE); Lars Frykberg, Uppsala (SE); Hans Lindmark, Uppsala (SE)

Correspondence Address: BIRCH STEWART KOLASCH & BIRCH, PO BOX 747, FALLS CHURCH, VA, 22040-0747, US

(21) Appl. No.: 10/269,017

(22) Filed: Oct. 11, 2002

Related U.S. Application Data
(63) Continuation of application No. 09/600,720, filed on Sep. 20, 2000.
Application No. 09/600,720 is a 371 of international application No. PCT/SE99/02448, filed on Dec. 21, 1999.

(30) Foreign Application Priority Data
Dec. 22, 1998 (SE) 9804491.0

(51) Int. Cl. G01N 33/554; G01N 33/569; A61K 39/02; C07H 21/04; C12P 21/02; C12N 1/21; C07K 14/315

(52) U.S. Cl. 424/190.1; 435/7.32; 435/69.3; 435/320.1; 530/350; 536/23.7; 435/252.3

(57) ABSTRACT

The present invention is concerned with a novel fibronectin-binding protein of Streptococcus equi, to a DNA fragment encoding this protein, to host cells and vectors containing said DNA fragment and to methods to produce said protein based on recombinant DNA technology.

The invention is also related to use of said protein in the preparation of a vaccine, to a vaccine containing said protein, to antibodies specific for said protein and to polyvalent antisera containing such antibodies.

This application is the national phase under 35 U.S.C. §371 of PCT International Application No. PCT/SE99/02448 which has an International filing date of Dec. 21, 1999, which designated the United States of America.

The present invention is generally related to a novel protein, methods to produce said protein and use thereof, e.g. for immunization purposes.

### BACKGROUND OF THE INVENTION

1. Field of the Invention

More specifically, the present invention is related to a novel fibronectin-binding protein derived from a bacterium belonging to the genus Streptococcus, to a DNA sequence encoding said protein, recombinant DNA methods for the production of said protein, and use of said protein per se or a fragment thereof as an immunogenic protein or antigenic polypeptide or peptide, e.g. for use as an active component in a vaccine, or to produce antisera.

2. Description of the Related Art

Streptococcal infections in horses are mainly caused by the species Streptococcus equi, which is classified as a Lancefield Group C Streptococcus and comprises two subspecies designated equi and zooepidemicus, respectively. Streptococcus equi subsp. equi which is virtually confined to horses is the causative agent of strangles, a world-wide distributed and serious disease of the equine upper respiratory tract. Since strangles is a highly contagious disease, not only infected animals but also all other members of an afflicted stud must be isolated for as long as up to three months.

S. equi subsp. zooepidemicus, is considered as an opportunistic commensal often occurring in the upper respiratory tract of healthy horses. However, after stress or virus infection, it can cause a secondary infection, which results in strangles-like symptoms. Moreover, subsp. zooepidemicus infects not only horses but also a wide range of other animals, like pigs, dogs, cats, and cows. Even human cases with infection due to subsp. zooepidemicus have been reported. This subspecies has been implicated as the primary pathogen in conditions such as endometritis, cervicitis, abortion, mastitis, pneumonia, abscesses and joint infections.

Although it is possible to treat and cure these streptococcal infections with antibiotics, such as penicillin, tetracycline or gentamicin, an effective prophylactic agent, that could prevent outbursts of such infections and obviate or reduce the risk for development of resistant strains associated with antibiotic treatment, would be appreciated.

However, although many attempts have been made to develop prophylactic agents such as vaccines against S. equi, at the present time no efficient vaccines or immunizing preparations are available, neither for the subspecies equi nor for the subspecies zooepidemicus.

Existing vaccines against strangles are based on inactivated, e.g. heat-killed, or attenuated strains of S. equi subsp. equi or acid extracts/mutanolysin enriched in M-protein(s), i.e. immunogenic protein(s) produced by S. equi. A vaccine against S. equi subsp. zooepidemicus based on an M-like protein is disclosed in U.S. Pat. No. 5,583,014.

Since the previously developed vaccines or immunizing preparations are hampered by side-effects and, moreover, provide insufficient protection, there is a need for efficient prophylactic agents, such as vaccines, that protect against S. equi infections and/or prevent spread thereof.

It is well known that attachment to eukaryotic cell surfaces is an essential step in the establishment of infection and colonization by bacterial pathogens. Accordingly, streptococcal surface proteins, that interact with and/or bind to different components of the Extracellular

Matrix (ECM) or plasma proteins of the host cell, are potential candidates for use as active component(s) for immunizing purposes.

This is illustrated by the vaccines based on M-like proteins mentioned above or disclosed in the literature, i. a. in WO 98/0561. The binding of fibrinogen and complement factor H to M-proteins is assumed to be important for the ability of streptococci to resist phagocytosis by polymorphonuclear leucocytes.

Another mechanism used by streptococci for attachment to host cells involves binding to the ECM component fibronectin (Fn) (3, 4). Binding between Fn-binding bacterial cell-surface proteins and immobilized Fn promotes internalization of streptococci by epithelial cells (1, 7, 11). Fibronectin is a dimeric glycoprotein found both in plasma and in a fibrillar form in the extracellular matrix. The main function of Fn is to mediate substrate adhesion of eukaryotic cells, which involves the binding of specific cell-surface receptors to certain domains of the Fn molecule (5). Furthermore, it also interacts with several other macromolecules, such as DNA, heparin, fibrin, and collagen (5).

Accordingly, several Fn-binding proteins from different streptococcal species have been cloned and sequenced previously. From S. equi, one Fn-binding protein has been cloned and characterized earlier, which is a Fn-binding cell-surface protein of subsp. zooepidemicus, that has been designated FNZ (9).

Recently, a novel gene encoding a Fn-binding protein has been cloned from S. equi subsp. equi. The encoded protein, is clearly distinguishable from the previously isolated streptococcal Fn-binding proteins inclusive of the FNZ protein.

The present invention is based on this novel protein originally derived from S. equi subsp. equi and its potential use for immunization purposes.

### BRIEF SUMMARY OF THE INVENTION

Generally, the present invention is directed to a protein having an amino acid sequence encoded by a nucleic acid sequence or gene, that forms a portion of the genome of S. equi subsp. equi, and which protein binds specifically to mammalian fibronectin.

The present invention is also directed to an isolated protein, specifically binding to fibronectin, such as mammalian, and specifically equine, fibronectin, and having an amino acid sequence as shown in SEQ. ID. NO. 1.

Moreover, the present invention is generally concerned with analogs or fragments of the present protein having fibronectin-binding-properties. For instance, a suitable fragment is comprised of the sequence of the present protein, that lacks the N-terminal signal sequence of the preprotein. A further suitable fragment of the present protein lacks a portion of said amino acid sequence, said portion comprising an amino acid sequence binding to a collagen-binding domain of fibronectin.

The present invention is also concerned with methods to produce the present protein, analogs or fragments thereof, which methods are based on DNA technology; and with nucleic acid sequences, and more specifically DNA sequences or fragments, intended for use in such methods, as well as use of said protein, analogs or fragments thereof for therapeutic purposes, such as immunizing purposes.

The novel protein has been termed SFS, and, accordingly, the corresponding gene is designated sfs. For the purpose of convenience, these terms are frequently used in the description.

### BRIEF DESCRIPTION OF THE DRAWINGS

In the following, the present invention is disclosed more in detail with reference to the drawings, where

FIG. 1 (A) shows a map of a clone designated pSFS62 with the gene sfs indicated.

FIG. 1 (B) shows a schematic presentation of protein SFS with the functional domains indicated. The bars correspond to the amino acid sequences of phagemid clones isolated by panning against Fn (S1-S4). Figures refer to the amino acid positions in protein SFS as shown in SEQ. ID. NO. 1 and the figures within brackets indicate the number of identical clones, that were isolated and sequenced.

FIG. 2 shows the results of Southern blot analysis of chromosomal DNA from ten streptococcal isolates. The DNA was digested by ApaI and separated by pulsed-field gel electrophoresis in duplicate. The radioactively labeled probe used corresponds to the gene sfs. Lanes: 1, subsp. zooepidemicus ZV; 2, S. dysgalactiae S2; 3, S. equisimilis 172; 4, subsp. equi Bd 3221; 5, subsp. equi Bd 995; 6, subsp. zooepidemicus DSM 20727^{T}; 7, subsp. zooepidemicus ATCC 53698; 8, subsp. equi CCUG 11664; 9, subsp. equi NCTC 9682^{T}; 10, S. pyogenes AW43. Molecular weight marker (concatamers of lamda) is indicated to the left.

FIG. 3 shows the results from inhibition assays related to Fn-binding. Cells of subsp. zooepidemicus ZV, subsp. zooepidemicus DSM 20727, subsp. equi Bd3221, and subsp. equi 640 were incubated with iodine-labeled Fn (hatched bars) and with a mixture of iodine-labeled Fn and protein SFS-E (striped bars). The bars represent means of duplicates and the standard deviation is indicated.

FIG. 4 shows the results from inhibition assays related to inhibition of binding between collagen and Fn with protein SFS. Collagen type I coated microtiter wells were incubated with Fn and a two-fold serial dilution of SFS. Bound Fn was detected by antibodies as described in Example 3. Points represent means of duplicates and the standard deviation is indicated.

### DETAILED DESCRIPTION OF THE INVENTION

More specifically, the present invention is directed to a fibronectin-binding (hereinafter abbreviated Fn-binding) protein, which has an amino acid sequence that can be expressed from a nucleic acid coding sequence, that can be isolated from and forms a portion of the genomes of S. equi, for instance subsp. equi.

According to a suitable embodiment, the present invention is directed to an isolated protein, specifically binding to fibronectin and having an amino acid sequence as shown in SEQ. ID. NO. 1 below, or a fragment or analog thereof.

| Met Arg Lys Thr Glu Gly Arg Phe Arg Thr Trp Lys Ser Lys Lys Gln: | SEQ. ID. NO. 1 |
|---|---|
| 1 5 10 15 | |
| Trp Leu Phe Ala Gly Ala Val Val Thr Ser Leu Leu Leu Gly Ala Ala | |
| 20 25 30 | |
| Leu Val Phe Gly Gly Leu Leu Gly Ser Leu Gly Gly Ser Ser His Gln | |
| 35 40 45 | |
| Ala Arg Pro Lys Glu Gln Pro Val Ser Ser Ile Gly Asp Asp Asp Lys | |
| 50 55 60 | |
| Ser His Lys Ser Ser Ser Asp Gln Pro Thr Asn His Gln His Gln Ala | |
| 65 70 75 80 | |
| Thr Ser Pro Ser Gln Pro Thr Ala Lys Ser Ser Gly His His Gly Asn | |
| 85 90 95 | |
| Gln Pro Gln Ser Leu Ser Val Asn Ser Gln Gly Asn Ser Ser Gly Gln | |
| 100 105 110 | |
| Ala Ser Glu Pro Gln Ala Ile Pro Asn Gln His His Gln Pro Gln Gly | |
| 115 120 125 | |
| Lys Pro Gln His Leu Asp Leu Gly Lys Asp Asn Ser Ser Pro Gln Pro | |
| 130 135 140 | |
| Gln Pro Lys Pro Gln Gly Asn Ser Pro Lys Leu Pro Glu Lys Gly Leu | |
| 145 150 155 160 | |
| Asn Gly Glu Asn Gln Lys Glu Pro Glu Gln Gly Glu Arg Gly Leu Pro | |
| 165 170 175 | |
| Gly Leu Asn Gly Glu Asn Gln Lys Glu Pro Glu Gln Gly Glu Arg Gly | |
| 180 185 190 | |
| Glu Ala Gly Pro Pro Ser Thr Pro Asn Leu Glu Gly Asn Asn Arg Lys | |
| 195 200 205 | |
| Asn Pro Leu Lys Gly Leu Asp Gly Glu Asn Lys Pro Lys Glu Asp Leu | |
| 210 215 220 | |
| Asp Gly Tyr Asn His Gly Arg Arg Asp Gly Tyr Arg Val Gly Tyr Glu | |
| 225 230 235 240 | |
| Asp Gly Tyr Gly Gly Lys Lys His Lys Gly Asp Tyr Pro Lys Arg Phe | |
| 245 250 255 | |
| Asp GLu Ser Ser Pro Lys GLu Tyr Asn Asp Tyr Ser Gln Gly Tyr Asn | |
| 260 265 270 | |
| Asp Asn Tyr Gly Asn Gly Asn Pro Asp | |
| 275 280 | |

The present invention is also related to proteins or polypeptides having an amino acid sequence as shown in SEQ. ID. NO. 1 containing deletions or substitutions of amino acids, such as fragments and analogs of the present protein having fibronectin-binding properties, suitably conserved, or specifically designed, Fn-binding properties. One such fragment or analog is comprised of the mature protein lacking the N-terminal amino acids no. 1 to 29 inclusive. Other fragments have en amino acid sequence corresponding to a portion of the amino acid sequence as shown in SEQ. ID. NO. 1 comprising a fibronectin-binding domain or an antigenic determinant or epitope. Still other fragments have an amino acid sequence corresponding to a portion of the sequence as shown in SEQ. ID. NO. 1, wherein an amino acid sequence binding to a collagen-binding domain of fibronectin (Fn) and comprising the amino acids (SEQ. ID. NO. 3) QGERGEAGPP, is deleted.

A further embodiment is concerned with a protein of the present invention having an amino acid composition of approximately 53 glycine residues, 39 serine residues and 38 proline residues evenly distributed in the protein and optionally 13 tyrosine residues in the C-terminal part of the protein.

Obviously, the present invention is concerned with a wild-type ptotein encoded by S. equi, that can be isolated and purified when recovered from said organism, as well as with a recombinant SFS protein as discussed above, said proteins having Fn-binding properties.

The present invention is also concerned with a nucleic acid sequence encoding the SFS protein or fragments or analogs thereof. Suitably, this sequence is a DNA sequence and contains an SFS coding sequence, such as the entire sfs gene, or a portion thereof encoding the SFS protein or a fragment or analog therof.

Accordingly, one embodiment of the present invention is related to a DNA sequence having a nucleotide sequence as shown in SEQ, ID. NO. 2 or to an equivalent thereof.

| | |
|---|---|
| SEQ. ID. NO. 2 | +TL, 44 |

| | |
|---|---|
| ATGAGAAAAA CAGAAGGACG TTTTCGCACA TGGAAGTCCA AAAAACAATG GCTATTTGCC: | 60 |
| GGTGCAGTGG GAGCTGCACT TGTCTTTGGA GGTTTATTAG GAAGTCTTGG TGGCTCATCC | 120 |
| CAGCAGCCAG TCAGCTCGAT TGGAGATGAC GATAAGTCGC ACAAGAGCTC ATCACCACCG | 180 |
| AAAAAGGATA ACTTGCAGCC TAAGCCTTCA GATCAGCCTA CTAATCGCCC GTCCCAGCCG | 240 |
| ACAGCAAAGA GCTCAGGTCA TCATGGGAAT CAACCTCACC AAGGAAATAG TAGTGGACAG | 300 |
| GCCTCAGAGC CTCAGGCTAT TCCTAATCAA GGGCTCAGAG GAGGTAACAG CTCTGGTTCA | 360 |
| GGTCATCACC ATCAGCCACA AGATCTAGGT AAGGATAATT CTAGCCCGCA GCCTCAACCA | 420 |
| AAGCCTCAGG GCAAAAAAGG CTTGAATGGT GAAAATCAGA AGGAACCGGA GCAAGGTGAA | 480 |
| CGAGGTTCAG GGTTGAGTGG TAATAATCAA GGCCGTCCTT CGCTTCCAGG CTTGAATGCA | 540 |
| GAGCAAGGTG AACGAGGTGA AGCCGGTCCC CCATCAACTC CGAATTTAGA TCCTTTAAAA | 600 |
| GGATTAGATG GAGAGAATAA GCCAAAGGAA GATTTAGACG GTAATGATGA ATCACCAAAA | 660 |
| CTTAAAGACG AACACCCCTA CAATCATGGT CGTCGCTATG AGGATGGATA TGGTGGCAAA | 720 |
| AAGCACAAAG GAGATTATCC TAAGCGAAAG GAATATAATG ACTATAGTCA AGGGTATAAT | 780 |
| GATAATTATG GAAATGGCGA TAGAGGTGGT AAGAGAGGAT ACGGCTATTC TTACAATCCC | 840 |
| GACTAA | 846 |

The present invention is also related DNA sequences having nucleotide substitutions, that do not change the encoded product or interfere with its expression. This is due to the well-known redundancy of the genetic code, i.e. more than one coding nucleotide triplet (codon) can code for or define a particular amino acid residue or a function, such as a stop codon function, etc. Thus, such functionally equivalent sequences are also encompassed by the present invention. Such equivalents may also arise due to spontaneous mutations.

Accordingly, when used to produce the present protein, such as a protein having the amino acid sequence shown in SEQ. ID. NO. 1, with methods based on recombinant DNA technology, the above DNA sequence may be modified to adapt to the codon frequency of the host organism used to produce the present protein, or fragments or analogs thereof.

The present invention is also concerned with a host cell comprising a DNA fragment or sequence of the present invention, e.g. a eukaryotic or, suitably, a prokaryotic host cell. A suitable prokaryotic host cell is derived from different strains of E. coli.

Furthermore, the present invention is concerned with a method to produce the present protein, fragments or analogs thereof comprising culturing a host cell containing the DNA sequence of the present invention, and isolating the expressed protein from the culture. Suitably this method also comprises purification of the expression product, such as affinity chromatography purification, conveniently based on use of “affinity tails”.

Thus, a suitable method comprises

(a) introducing a DNA fragment of the present invention into an expression vector;

(b) introducing the said vector, which contains the said DNA fragment, into a compatible host cell;

(c) culturing the host cell provided in step (b) under conditions required for expression of the product encoded by said DNA fragment; and

(d) isolating the expressed product from the cultured host cell.

Preferably, said method further comprises a step (e), wherein the isolated product from step (d) is purified, e.g. by affinity chromatography, such as Fn-affinity chromatography, or with the use of “affinity tails” as is well-known in this field of art.

The present invention is further concerned with a vaccine comprising the present Fn-binding protein or a fragment or an analog thereof as an antigenic or immunogenic component. This vaccine is intended for use as a vaccine protecting against infection with any one of the two subspecies equi and zooepidemicus of S. equi.

A further embodiment of the present invention is concerned with a vaccine as defined above, that protects horses against strangles caused by S. equi subsp. equi infection. Suitably, such a vaccine could protect also against infection with subsp. zooepidemicus.

The vaccine of the present invention is suitably a subunit vaccine. Moreover, the vaccine may be comprised of a cocktail of antigenic and/and or immunogenic components comprising the present protein or an analog or a fragment thereof as one such component.

The present invention is also concerned with use of the present Fn-binding protein, analogs or fragments thereof in the preparation of a vaccine protecting against S. equi, for instance a vaccine as disclosed above.

Furthermore, the present invention is also concerned with the production of antibodies raised against the present protein, analogs or fragments thereof, with fragments of such antibodies and with the production of antisera. Antibodies and/or antisera could be produced by in vivo administration, e.g. injection, of an antigen comprising the said protein, an analog or a fragment thereof, to a host to elicit an immune response in said host, and recovering antiserum thereby produced in said host and, optionally, recovering or isolating antibodies contained in said antiserum or in other body fluids from said host. Not only polyclonal but also monoclonal antibodies could be produced in accordance with well-known methods.

### Experimental Part

Bacterial strains, plasmids, and growth conditions. Ninety-eight clinical isolates of S. equi (50 isolates of subsp. equi strains and 48 isolates of subsp. zooepidemicus strains) collected from different parts of Sweden between 1982-1996 were together with the streptococcal control strains S. dysgalactiae S2 and S. equisimilis 172 obtained from the National Veterinary Institute, Uppsala. The S. pyogenes strain AW-43 was a kind gift from Dr G. Lindahl, Lund University. The plasmid pUC19 was used for cloning purposes and pGEX-5X-2 (Pharmacia Biotech, Uppsala, Sweden) for facilitating purification of proteins. Phagemid pG8SAET (19) was used for purification of protein SFS and for construction of the phage display library. Streptococcal strains were grown on horse blood agar plates or in Todd-Hewitt broth (Oxoid, Basingstoke, UK) supplemented with 0.5% yeast extract (THY). E. coli strains were cultured in Luria-Bertani (LB) medium supplemented in appropriate cases with 50 μg of ampicillin per ml.

As regards the isolates of S. equi, it is known that isolates of subsp. equi are serologically and genetically very homogeneous whereas isolates of subsp. zooepidemicus display a high degree of heterogeneity (2, 8, 10, 13).

### EXAMPLE 1

### Cloning and Isolation of a Gene sfs Encoding the Fn-binding Protein SFS.

A. Construction of a phagemid library. A shotgun phage display library was constructed from subsp. equi Bd 3221 essentially as described by Jacobsson and Frykberg (6). Briefly, chromosomal DNA of this strain was isolated as described earlier (9) and subsequently purified and fragmented by sonication.

The obtained fragments were treated with T4 DNA polymerase to generate blunt ends and subsequently ligated into SnaBI-digested and dephosphorylated pG8SAET vector. Approximately 4.5×10⁶ ampicillin-resistant transformants were obtained after electrotransformation of the ligated material into E. coli, TG1 cells.

Twenty randomly picked transformants were all shown to contain inserts. Cells from an overnight culture of the transformants were infected with helper phage R408 and poured together with soft agar onto LA+ampicillin plates (LB medium supplemented with 1.5% agar and 50 μg ampicillin/ml) and incubated overnight. Phage particles were eluted from the soft agar by addition of LB and vigorous shaking. The suspension was centrifugated and the supernatant sterile filtrated. The titer of the library was determined to 7×10¹⁰ CFU/ml.

This library was used in the following Section B, wherein phage particles containing inserts related to Fn-binding properties are identified.

B. Panning of the phagemid library. Microtiter wells (Maxisorp, Nunc, Copenhagen, Denmark) were coated with human Fn (Sigma, St. Louis, Mo.) at a concentration of 100 μg/ml in 50 mM sodium carbonate, pH 9.7. The wells were blocked with PBS-0.05% Tween 20 (PBS-T) containing casein (0.1 mg/ml). After washing the wells with PBS-T, the library from Section A above was added to the wells. Thereafter, the wells were extensively washed with PBS-T and then eluted with 140 mM NaCl, 50 mM Na-citrate (pH 2.0).

To obtain transformed cells containing the Fn-binding insert, eluate was collected, neutralized, infected with E. coli TGI cells and spread on LA plates containing ampicillin. Next day, approximately 1,500 colonies were pooled and after infection with helper phage R408 the sample was mixed with soft agar and poured out on LA plates. After incubation overnight, the phagemid particles were extracted and subjected to another round of panning. After the first panning, 41% of the colonies were found to bind Fn and after the second panning all 180 colonies were positive for Fn-binding.

C. Screening for Fn-binding clones containing an insert encoding SFS. In this section, the gene encoding the novel Fn-binding protein SFS was screened for based on the knowledge that the previously disclosed fnz gene isolated from subspecies zooepidemicus and encoding the Fn-binding protein FNZ is present also in the genome of subspecies equi. Thus, a negative screening test was used, wherein cells containing inserts related to Fn-binding, i.e. cells positive when screening with a rabbit anti-Fn antibody, but not containing inserts related to Fn-binding of FNZ, i.e. those of the positive cells that were negative when the fnz gene is used as a screening probe, were selected and presumed to contain SFS-related inserts.

From each panning performed in section B, 180 colonies were transferred in triplicate to LA plates and incubated overnight. The following day, one plate was stored (masterplate) and the colonies from the two remaining plates were transferred to nitrocellulosa filters and incubated for two hours.

Cells from one filter were lysed using chloroform vapor and after blocking the filter with PBS-T supplemented with casein (0.1 mg/ml), it was incubated with human Fn (1 μg/ml; Sigma) for 2 h, and after washing, a rabbit anti-Fn antibody (diluted 1/1000; Sigma) was added. After 1 h of incubation and washing, the filter was incubated for additional 1 h with a HRP-labeled secondary antibody (diluted 1/1000; Bio-Rad, Richmond, Calif.). Reactive bands were visualized by using 4-chloro-1-naphthol (Serva, Heidelberg, Germany).

The second filter was subjected to colony hybridization essentially as described in Sambrook et al. (22) with use of a radioactively labeled probe that covered the entire fnz gene and was generated by PCR amplification of chromosomal DNA from subsp. zooepidemicus strain ZV using the primers:

| | | |
|---|---|---|
| 5-fnz, 5′-CGGGATCCCTATTACACATTCTCATCTCATAT | (positions 19-42) and | (SEQ. ID. NO. 4) |
| 3-fnz, 5′-GGAATTCCAGAAAGCCCGCCTGTAAAC | (positions 1954-1935). | (SEQ. ID. NO. 5) |

The indicated positions in the respective primers correspond to the published sequence of the genefnz (9).

In these colony hybridization tests, 41% of Fn-binding clones from the first panning and 30% from the second panning hybridized against the frz gene from subsp. zooepi-demicus ZV used as a probe.

D. Cloning and isolation of the gene sfs. Clones from Section C, displaying Fn-binding activity but negative in the colony hybridization assay, were selected as candidate sfs-gene-containing clones. Accordingly, these clones were sequenced using thermo sequence dye terminator cycle sequencing pre-mix kit (Amersham) and the ABI Model 377XL DNA sequencer. Computer programs from the PCGENE, DNA, and protein sequence analysis software package (Intelligenetics, Inc., Mountain View, Calif.) were used to record and analyze the sequence data.

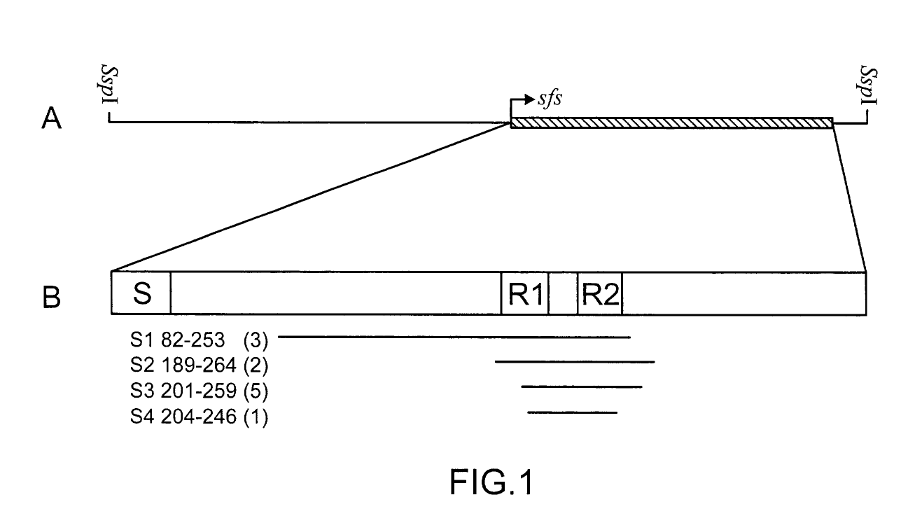

Altogether, eleven Fn-binding but fnz negative clones were analyzed and found to contain inserts identical to one of four different types of inserts, all with overlapping sequences and an open reading frame. These four different phagemid clones designated S1-S4 are shown in FIG. 1B. Based on the overlapping sequences, primers were designed and used to generate a probe consisting of the complete sfs gene using PCR amplification as disclosed in the section concerned with Southern blots below.

To isolate the complete gene encoding the Fn-binding activity, Southern blot analysis of restriction enzyme digested chromosomal DNA of subsp. equi Bd 3221 was performed as disclosed below and revealed that a 2.6 kb SspI fragment contained sfs. Accordingly, fragments of this size were purified from a preparative agarose gel and ligated into pUC 19. The ligation mix was electroporated into E. coli and transformants were screened for Fn-binding activity as described above. Among several positive clones one, designated pSFS62, was selected and the insert sequenced.

This clone, pSFS62, had an open reading frame of 1,035 bp, from which the phagemid sequences were found to originate (FIG. 1 and SEQ. ID. NO. 2). The open reading frame is preceded by sequences typical for promoter and ribosome-binding sites (not shown) and is followed by sequences (not shown) resembling a transcriptional termination, suggesting that the gene is translated from a monocistronic messenger. The SFS-coding nucleotide sequence of the sfs gene is shown in SEQ. ID. NO. 2.

Southern blots. The Southern blot analysis referred to above and further below, was performed according to the following. Agarose imbedded chromosomal DNA digested with ApaI was resolved on 1.2% SeaKem GTG agarose gel (FMC, Rockland, Me.) in 0.5×TBE buffer by PFGE using a Gene Navigator (Pharmacia Biotech, Uppsala, Sweden) as earlier described (10). The DNA was transferred to nylon filters (Hybond-N+, Amersham) by vacuum blotting (VacuGene XL, Pharmacia Biotech) in accordance with the manufacturer's protocol. After cross-linking, the filters were prehybridized for 2 h at 65° C in 6×SSC, 3 × Denhardt's solution, and 0.5% SDS and subsequently incubated with the radioactively labeled sfs probe overnight, using the same conditions. The membranes were washed 3×20 min at 65° C with 0.2×SSC, 0.1% SDS and subjected to autoradiography.

The probe sfs was generated by PCR amplification of chromosomal DNA from subsp. equi Bd 3221 using the primers:

(SEQ. ID. NO. 6)

fs5, 5′-ACAAGCCATGGAGCACTTGTCTTTGGAGGT and

(SEQ. ID. NO. 7)

fr4, 5′-GTCGGGATTGTAAGAATAGCC.

The single band obtained after agarose gel electrophoresis was purified and random-primed.

### EXAMPLE 2

### Construction and Purification of SFS as a Fusion Protein.

The purified PCR-fragment from Example 1 encoding the mature protein of SFS, and described under Southern blots in Example 1, Section D, was digested with NcoI and ligated into SnaBI-NcoI opened pG8SAET. This vector encodes a 13 amino acid peptide tag (E-tag) which facilitates the purification of the recombinant protein using a HiTrap Anti-E tag column (Pharmacia Biotech). The recombinant protein SFS-E was purified from the periplasmic space according to the manufacturer's protocol.

After cleaving from the E-tag, the SFS protein was obtained having a calculated molecular mass of 40 kDa. The charged amino acids, followed by a stretch of hydrophobic residues in the N-terminal end of the protein, indicate a signal sequence and by the method of von Heijne (14) a possible signal sequence cleavage site was found between amino acids 29 and 30, resulting in a mature protein with a calculated molecular mass of 36 kDa. The isolated Fn-binding phagemid clones contained inserts originating from the central part of the protein, where two repetitive sequences of 21 residues, called R1 and R2, resp., are situated (FIG. 1). Three amino acids were found to dominate the composition of protein SFS, 53 residues are glycines (14.4%), 39 serines (10.6%), and 38 prolines (10.3%). These three amino acids are evenly distributed in the protein in contrast to the 13 tyrosine residues which occur only in the C-terminal part of the protein. Protein SFS does not contain any sequence motifs known to mediate attachment to the bacterial cell-wall.

### EXAMPLE 3

### Inhibition Assays

Cells from overnight cultures of streptococci were collected by centrifugation, washed in PBS, and suspended in PBS-0.2% Tween 20 to an optical density at 600 nm of 0.2. In cases of inhibition, 25 nM of affinity purified fusion protein SFS-E was preincubated 15 min with 16 pM of ¹²⁵I-labeled human Fn (91,061 cpm) and thereafter bacteria (500 μl) were added. After two hours incubation at room temperature, the mixtures were centrifuged and the supernatants removed. The radioactivity associated with the pellets was quantified in a gamma counter (LKB Wallac, Turku, Finland). Radioactivity (808 cpm) recovered from a control (tubes that contained no streptococci) was subtracted from each test.

### EXAMPLE 4

### Expression of the sfs Gene

RNA was extracted from S. equi cells by using the Blue FastRNA kit (Bio 101, Vista, Calif.) according to the manufacturer's protocol. RNA concentration was determined spectrophotometrically and by visual estimation of the rRNA bands on an agarose gel. RNA (10 μg) was loaded on a formaldehyde-containing agarose gel. RNA was transferred by vacuum-blotting to a positively charged nylon filter (Hybond-N+, Amersham) and cross-linked. Further steps were performed as described for Southern blots above with the exception that ssDNA was added to the pre-hybridization and hybridization solutions.

### EXAMPLE 5

### The Ability of SFS to Inhibit the Binding Between Collagen and Fn.

For the enzyme-linked immunosorbent assay (ELISA), polystyrene 96-well microtiter plates were coated for one hour with collagen type I from calf skin (Boehringer, Mannheim, Germany) in PBS. The wells were blocked for one hour with PBS-T supplemented with casein (0.1 mg/ml) and then washed four times with PBS-T. The fusion protein SFS-E was diluted in a two-fold serial and added to the wells together with 0.2 ng of Fn. After 2 h incubation, the wells were washed and a rabbit anti-Fn antibody was added and allowed to bind for 1 h. Finally, the wells were incubated for 1 h with a secondary HRP-labeled antibody. After washing, bound material was quantified by using tetramethylbenzidine (Boehringer) and a microplate reader (Bio-Tek Instruments, Vinooski, Vt.). Measurement was done at a wavelength of 450 nm. Absorbancy in wells without added fusion protein was set to 100% and absorbancy in wells where Fn had been excluded was set to 0%.

### RESULTS

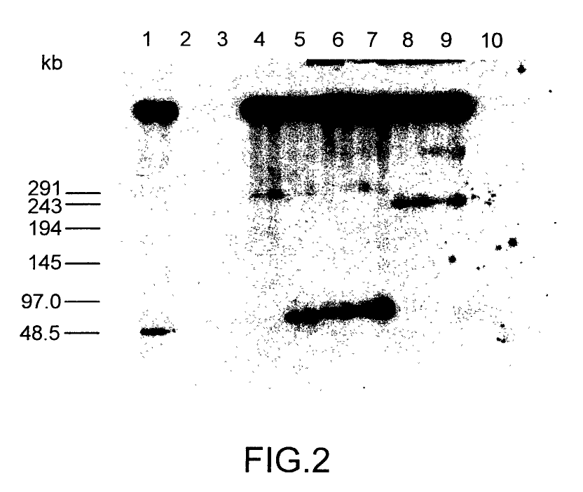

I. The gene sfs is generally present in isolates of subsp. equi. Southern blots performed as disclosed above revealed that a [2 P] dATP-labeled probe, corresponding to the gene sfs, hybridized to all the 50 subsp. equi and to 41 out of 48 subsp. zooepidemicus isolates tested. The results from the hybridization analysis are, for a selected number of strains, shown in FIG. 2. No significantly weak signal, that could not be explained by less chromosomal DNA on the gel, was detected for any of the positive S. equi isolates. The seven isolates of subsp. zooepidemicus that were sfs negative could not be related to each other, considering symptoms, temporal, and geographical origin. Furthermore, the seven negative isolates were obtained from different species, horses (n=4), cows (n=2), and dog (n=1). The sfs probe did not hybridize to any of the three control strains of other streptococcal species (FIG. 2).

II. Protein SFS displays sequence similarity to both collagen and a potential cell-wall protein of S. pyogenes. Collagen sequences gave highest scores when searching the database Swissprot for SFS-like sequences. The similarity was evenly distributed through protein SFS, and the main reason for the high score is the high content of glycine, serine, and proline, i.e. residues which are also common in collagen. However, a more pronounced similarity was seen for the Fn-binding domain of SFS against collagen. A sequence comparison was also done against the Oklahoma S. pyogenes genomic sequence database, which at the time of search consisted of 98% of the S. pyogenes genome. SFS aligned best against a database sequence which besides high content of glycine and proline residues also displayed the motif (SEQ. ID. NO. 8) QGERGETGP. Eight of these nine residues are present in the Fn-binding domain of SFS. Similar motifs are also present in chains of collagen. Alignment of these sequences are shown in the following Table I. At a closer study of the aligned S. pyogenes sequence, it was found that the aligned motif is situated in the middle of a potential gene, encoding a typical streptococcal cell-surface protein. This statement is based on the following: (i) promoter sequences and a putative ribosome-binding site are present adjacent to an open reading frame, (ii) in the C-terminal part there is a proline-rich domain with the cell-wall anchoring motif (SEQ. ID. NO. 9) LPXTGX, (iii) the LPXTGX motif is directly followed by a stretch of 23 hydrophobic residues and the open reading frame is terminated by six residues whereof three are charged, and (iv) a potential hairpin loop is situated 38 bp downstream the stop codon. However, a start codon in an acceptable distance to the ribosome binding site could not be found.

TABLE I

| | | |
|---|---|---|
| SFS | (SEQ. ID. NO. 10) | QGERGEAGPP |
| S. pyogenes | (SEQ. ID. NO. 11) | QGERGETGPA |
| Collagen α2 (I) 711 | (SEQ. ID. NO. 12) | PGERGEVGPA |
| Collagen α1 (I) 991 | (SEQ. ID. NO. 13) | SGERGPPGPM |
| Collagen α1 (II) 329 | (SEQ. ID. NO. 14) | PGERGRTGPA |
| Collagen α1 (III) 797 | (SEQ. ID. NO. 15) | PGERGETGPP |
| Collagen α1 (IV) 319 | (SEQ. ID. NO. 16) | QGEKGEAGPP |

In this table, alignment of amino acid sequences from different types of collagen and the potential cell-surface protein from S. pyogenes to a motif present in the Fn-binding domain of SFS is illustrated. The figures indicate the number of the first amino acid from the collagen sequences. Bold letters indicate identical residue to the SFS motif.

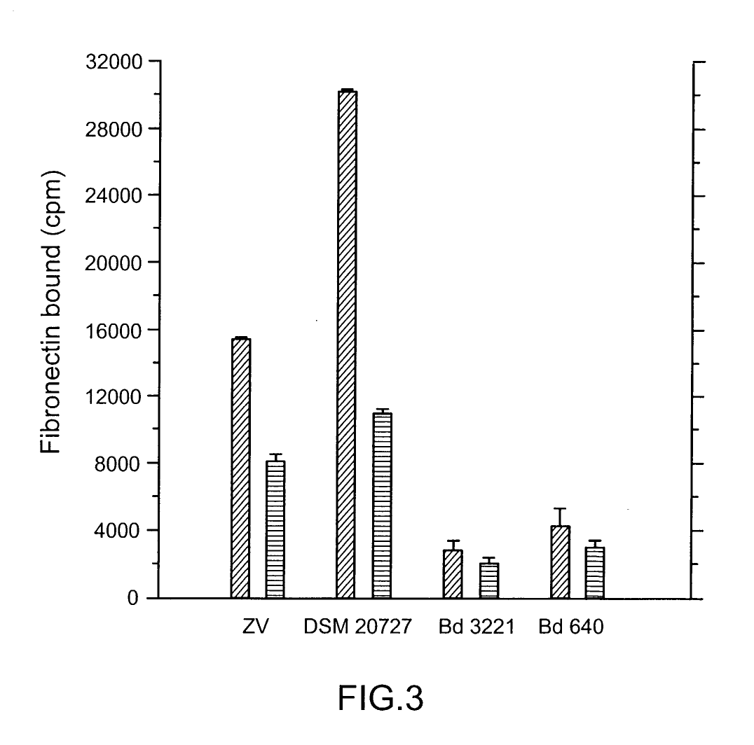

III. Inhibition of binding between Fn and cells of S. equi. Recombinant protein SFS was purified by using affinity tails and the purified protein was found to bind Fn in a Western blot assay (data not shown). Before adding iodinated Fn to cells of S. equi the labeled Fn was, in appropriate cases, preincubated with SFS in a molar ratio of 1:1,500. After incubation the cells were collected by centrifugation and after removing the supernatant, the radioactivity bound to the pellets was measured. From the results from the inhibition experiments shown in FIG. 3 it is evident that the protein SFS has, for both subspecies, an inhibitory effect, although the two subsp. equi strains bind considerable less Fn compared to the two subsp. zooepidemicus strains

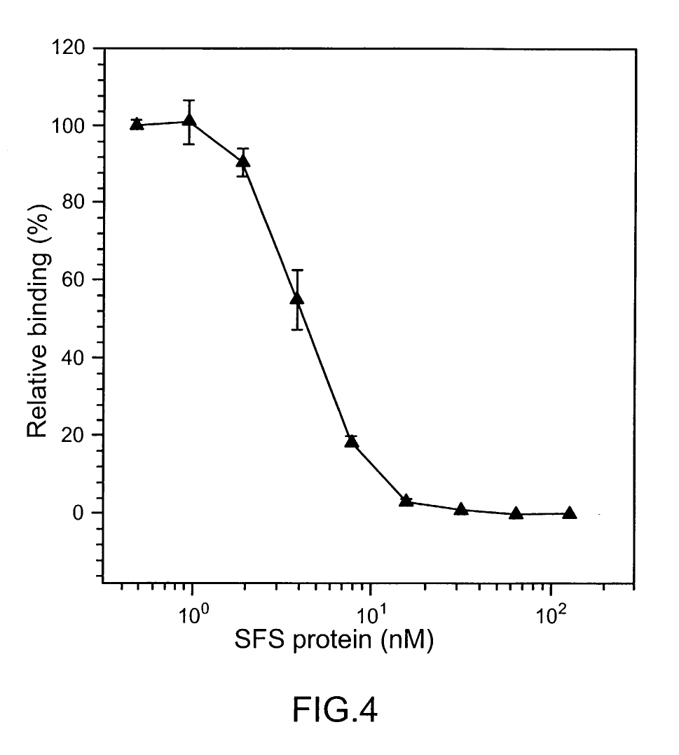

IV. Protein SFS inhibits the binding between Fn and collagen. The similarity between protein SFS and collagen suggested that these proteins might bind to the same site on the Fn molecule. In order to investigate this, microtiter wells coated with collagen were incubated with a mixture of Fn and a serial dilution of protein SFS. Bound Fn was detected by an anti-Fn antibody and as seen in FIG. 4, protein SFS inhibits the binding in a concentration dependent way. In a similar assay, the previously known protein FNZ did not inhibit the binding between Fn and collagen (data not shown). Furthermore, protein SFS did not inhibit the binding between the said protein FNZ and Fn, and the protein FNZ did not inhibit the binding between protein SFS and Fn. Protein SFS does not bind collagen. This was tested in order to control that the inhibition of binding between Fn and collagen by protein SFS is dependent on the binding of protein SFS to Fn and not to collagen. Taken together this suggests that protein SFS and the previously known protein FNZ have clearly separate binding sites on the Fn molecule and that protein SFS binds to the 30-40 kDa collagen-binding domain of Fn.

In the following Example 6, the potential use of the present protein as a vaccine is illustrated in a test wherein the immunogenic properties of the present novel protein are confirmed.

Example 6. Immunogenic properties of Protein SFS. Affinity purified recombinant protein SFS (Example 2) was, under reducing conditions, subjected to SDS-PAGE on a precasted 8-25% gradient-gel using the PHAST system (Pharmacia Biotech, Sweden). The molecular weight markers used were obtained from BioRad, CA, USA. After electrophoresis was completed, a nitrocellulose (NC) filter (Hybond C, Amersham, UK) previously soaked in PBS was put on the gel and the temperature raised to 45° C. After 45 minutes, the NC-filter was wetted with 1 ml PBS, and removed and placed in 15 ml PBS-T containing casein (0.1 mg/ml) for 1 hour (with two changes of PBS-T casein solution) at room temperature under gentle agitation.

The gradient-gel was after transfer removed and stained with Coomassie-blue using the PHAST system. The NC-filter was removed and incubated in 5 ml PBS-T casein solution containing 5 μl serum from a horse which previously had got the diagnosis strangles and found to be a carrier of S. equi. After 2 hours incubation at room temperature, under gentle agitation, the filter was extensively washed with PBS-T and incubated in 5 ml PBS-T casein solution containing rabbit anti horse antibodies (Nordic Immunology, Netherlands) at a dilution of 1:1000. After 1 hour of incubation at room temperature, under gentle agitation, the filter was extensively washed with PBS-T and incubated in 5 ml PBS-T casein solution containing horseradish peroxidase labeled goat anti rabbit IgG (BioRad) at a dilution of 1:1000. After 1 hour of incubation at room temperature, under gentle agitation, the filter was extensively washed with PBS-T and PBS.

To visualize the bound IgG conjugate, the filter was transferred to a solution containing a substrate for peroxidase (containing 25 ml PBS +6 ml 4-chloro-1-naphtol (Sigma, USA, 3 mg/ml in methanol)+20 μl H₂O₂ (35%). After about 15 minutes, the degree of color was measured by eye. The bands appearing on the NC-filter and the bands appearing on the corresponding Coomassie-blue stained PAGE were compared.

The obtained results clearly showed that (i) the recombinant produced SFS protein was recognized by antibodies present in the serum from the horse with strangles, and (ii) no bands were seen on the NC-filter in the lane with the different molecular weight markers. Thus, this means that protein SFS is expressed by S. equi during the infection process and that this protein is immunogenic

### REFERENCES

1. Cue, D., P. E. Dombek, H. Lam, and P. P. Cleary. 1998. Streptococcus pyogenes serotype M1 encodes multiple pathways for entry into human epithelial cells. Infect. Immun. 66:4593-4601.

2. Galan, J. E., and J. F. Timoney. 1988. Immunologic and genetic comparison of streptococcus equi isolates from the united states and europe. J. Clin. Microbiol. 26:1142-1146.

3. Hanski, E., and M. G. Caparon. 1992. Protein F, a fibronectin-binding protein, is an adhesin of the group A streptococcus Streptococcus pyogenes. Proc. Natl. Acad. Sci. USA 89:6172-6176.

4. Hanski, E., P. A. Horwitz, and M. G. Caparon. 1992. Expression of protein F, the Fibronectin-binding protein of Streptococcus pyogenes JRS4, in heterologous streptococcal and enterococcal strains promotes their adherence to respiratory epithelial cells. Infect. Immun. 60:5119-5125.

5. Hynes, R. O. 1990. Fibronectins. Springer Verlag, New York.

6. Jacobsson, K., and L. Frykberg. 1996. Phage display shot-gun cloning of ligand-binding domains of procaryotic receptors approaches 100% correct clones. BioTechniques 20:1070-1081.

7. Jadoun, J., V. Ozeri, E. Burstein, E. Skutelsky, E. Hanski, and S. Sela. 1998. Protein F 1 is required for efficient entry of Streptococcus pyogenes into epithelial cells. J. Infect. Dis. 178:147-158

8. Jorm, L. R., D. N. Love, G. D. Bailey, G. M. Bailey, and D. A. Briscoe. 1994. Genetic structure of populations of P-haemolytic Lancefield group C streptococci from horses and their association with disease. Res. Vet. Sci. 57:292-299.

9. Lindmark, H., K. Jacobsson, L. Frykberg, and B. Guss. 1996. Fibronectin-binding protein of Streptococcus equi subsp. zooepidemicus. Infect. Immun. 64:3993-3999.

10. Lindmark, H., P. Jonsson, E. Olsson Engvall, and B. Guss. 1998. Pulsed-field gel electrophoresis and distribution of the genes zag and fnz in isolates of streptococcus equi. In Press.

11. Molinari, G., S. R. Talay, P. Valentin-Weigand, M. Rohde, and G. S. Chhatwal. 1997. The fibronectin-binding protein of Streptococcus pyogenes SfbI, is involved in the internalization of group A streptococci by epithelial cells. Infect. Immun. 65:1357-1363.

12. Nilsson, M., L. Frykberg, J. I. Flock, L. Pei, M. Lindberg, and B. Guss. 1998. A fibrinogen-binding protein of Staphylococcus epidermidis. Infect. Immun. 66:2666-2673.

13. Skjold, S. A., P. G. Quie, L. A. Fries, M. Barnham, and P. P. Cleary. 1987. DNA fingerprinting of streptococcus zooepidemicus (Lancefield group C) as an aid to epidemiological study. J. Infect. Dis. 155:1145-1150.

14. von Heijne, G. 1986. A new method for predicting signal sequence cleavage sites. Nucleic Acids Res 14:4683-4690.

Nucleotide Sequence Accession Number: The nucleotide sequence of the fnz gene (9) is available from the EMBL sequence data bank under accession number X99995. The complete gene sequence (SEQ. ID. NO. 17) and the deduced amino acid sequence (SEQ. ID. NO. 18) of the protein FNZ are shown below:

| −35 −10 RB | |
|---|---|
| AAAGAAATCACCTTAAAACTATTACACATTCTCATCTCATATGATATAGTTATTCACCTTTCCTAACCCAAAACATATTAAAAACTTAATATACGGAG | 120 |
| | 4 |

S
AGAAGACACTTCAAAACAAAAT
L K T K

| | |
|---|---|
| ↓ | |
| CATTTAGAAAGGTACTGACGACGTCAGCTACCTGTATTGTGCTGCCAACAACCTTT [symbol] ACCAACCCTAC [symbol] TCTGGGCAGAGCAGCTTTATTAT | 240 |
| S F A K V L T T S A T C I V L A T S F A G G T L A V W A E Q L Y Y | [symbol] |

GGGT [symbol] AATCATCCAACCACAC
C W N D G T R

| | |
|---|---|
| AAAGTTCGCCATATTTTTTGTACGTATCCCCTAAAAATCCTCCAACCCTGAATTAAAAGACGAGTATGTTGTTTATTGCTTTAACAAAAAATTCTATT | 360 |
| Q S S F Y F L Y V S P K N A P K R E K D E Y V V Y C F N K K L Y | 8 [symbol] |

CCCCAGATCAATGGGAATCTA
W P D Q W E S

| | |
|---|---|
| TATACACCAATTTTAATGACATCACATCTCCATATAACCATTACCTGTATATGAGAAAAAACTAGGATATGATATTTAAACAATATCCTCCA | 480 |
| I Y S N F N D I R S P Y N D L F V Y E K K L G Y D G I F K Q Y A P | 124 |

CATTACAAAAAAGATATTAGTG
D Y K K D I S

| | |
|---|---|
| ATATTCCAACTCCTTTCCTCCCACTTTTAAGTAATGGATACCCCA [symbol] AACAAGTCACAACTATCAACTACCTACCATTTAAATAATGATTCTT [symbol] TAGA | 600 |
| D I A S A L V A V L S N G Y P T N K S Q L S T S Y H L N N D S S R R | 164 |

AAAGTTACTCAATTAGCCATTT
K V T Q L A I

| | |
|---|---|
| [symbol] TATTTTACTCATAGTTTAACAAAAGAATACCTTAAAGATACGGGTGGTTATAACTTAAACGATATCCAAAAAAAACCTTTACATTTTTTAAT [symbol] AGT | 720 |
| W Y F S D S L T K E Y L K D T C C Y N L N D M E K K A L D F L I [symbol] | 204 |

AAAGGAGAGGATTCTAAGCTTA
K C E D S K L

| | |
|---|---|
| AATCACACCACACTAATTACTCATTCCATATTTATCTTTATCAAAGTGGCGGGCATGACCATATGAAAGATTACCAAAATCTTCTCGCCTCTACCTTA | 840 |
| K S [symbol] O S N Y S L D I Y V Y C [symbol] G C H D H M K D Y Q N L L G S T L | 244 |
| → CP | |

ATTCCTAAAGAACCGCTAAAGC
I P K E P L K

| | |
|---|---|
| → E1 | |
| CTCAGCTAGGTCCTTTTAGTCCACATAATCCAAATCCATTAA [symbol] TTGAAGGAGGATCATCAGGTTCACAAGAAACTAATCAACATCCTAACAA | 960 |
| P Q L C C P S G H N G N G L S G L E G G S [symbol] G S Q E T N E D G K K | [symbol] |

GGACTTATAGGTTTCCATGGAG
G L I C P H C

| | |
|---|---|
| → [symbol] 2 | |
| GACTCTCAGGAAGCGAGGGCAAACGAGATCCTTTGCCAGGATTGAACCCTCACCCTGGTGCACCTGATACACCTCAAAAGCCTAATGATCCATTGCAA | 1080 |
| G L S G S E G K R D F L P C L K G R A G A P D T P Q K P N D P L Q | 324 |

GCTCTTCAACCCGGTAACTCTC
G L E G G N E

| | |
|---|---|
| → A2 | |
| CTATAGTACAACAAAACTATGGTAGTACCCAAGGATATCATGGTCAATCAGGCATTCTTGAGCAAACCCAACATACTAACCCACCTGGTATCATACTA | 1200 |
| P I V E Q N Y G S T E G Y H G Q S C I L E E T E D T N F F G I I L | 364 |

→ R1
GGCGGCTCACCAAATGTCGAAA
C G S G N V E

| | |
|---|---|
| → R2 | |
| CGCATGAACATACTACAACCCTCATCTGATCCCCATCGGCGGGGGTCTAGCTGGCGAATCAGGAGAACGACACCTAAACCACCACAAACCGGTGGG | 1320 |
| T H E D T E N D M L M G I G G G L A G E S C E T T P K P G Q T G G | 404 |

CAAGGACCAGTCATCGAGACAA
Q C P V I E T

| | |
|---|---|
| → R3 | |
| CAGAGGATACACAAAAAGGCATGTCTGCACAATCTGGTGGCACTAT [symbol] AGTCAGAAAACACCAAAAAGCCGGAGGTCATGATTGGTGGTCACCCACAA | 1440 |
| T E D T Q K C [symbol] [symbol] C Q [symbol] C G T I [symbol] [symbol] E N T K K P E V M I G G Q G Q | 444 |

ACCATCGAGACAACAGAGGACA
T I E T T E D

| | |
|---|---|
| → R4 | |
| CACAAAAAGGCATGTCTGGACAATCTGGCGGTACTATCGAGTCAGAGCACACTAACAAACCTCAGGTCATCATTGGTGGTCAGGGACAAATCATTGAC | 1560 |
| T Q K G M E G G E C C T I E S E D T K K P E V M I G G Q G Q I I D | 484 |

TTCTGAAAACACCATCAG
P S E N T G S

| | |
|---|---|
| → R5 | |
| CTATGTCTCCCCACTCTGGTCACACTACGGTAATTGAGGATACCAAGAAGTCTGACATAATCATTCCTCCCCAACCACAAATCATGGACTTCTCTGAG | 1660 |
| [symbol] M S G Q S G D T T V I E D T K K S E I I I G G Q G Q I I D P E E | 524 |

GATACTCACCCGGGTATGTCTG
D T Q P G M S

| | |
|---|---|
| → W | |
| GTCAATCTGGACGCACTACAATTGTGGAAGACACCAAGAAGCCGAGACCTAAGCCTAAACCTGCACCTGCGCCAATTCTTAATCACCAAAAACCTAAC | 1810 |
| C Q E C C T T I V E D T K K F T F K F K P A P A P I V N D E K F N | 554 |

AAAGGCACTCATCTCCCACAGA
K G T H L P Q

| | |
|---|---|
| → [symbol] | |
| CAAGTGATATGAAGCAACTCACCCTAAGCATCATCCCTGCAATCTCAATCTCAATGCTGCTTGTCCTATGTCTGTCTCTATTCAAGCGACCATCTAAAAAAGAG | 1920 |
| T E D M K C L T L S I I G A M S M L L V L C L S L F K R D S K K D | 597 |

TAAGCAACACTGACAAGTGATC
*

ATTATTGTACGGACGTTTACAGGCGGGCTTTCTCCCCTCTTAAAACACCGTACAACACCAAAACGGTCTAAATAGAAATATCCCTCAAGGCTTAGACA

ATCGCTCTAGCCCTTGGGGGCT

TTTTTTCACATTTATAATAGAAGAGAGCAAGCAGTGACACTTTCTTTTTGCCCTATATCTGTTAACACTAGCGGTATGTGCAGTTAC

1. An isolated protein, an analog thereof, or a fragment thereof, having an amino acid sequence encoded by a nucleic acid sequence, that can be isolated from and forms a portion of the genomes of Streptococcus equi, which protein can be expressed from said nucleic acid sequence and which protein binds specifically to mammal fibronectin, provided that the amino acid sequence shown in SEQ ID NO. 18 is excluded.

2. An isolated protein specifically binding to fibronectin and having an amino acid sequence as shown in SEQ. ID. NO. 1, or an analog or fragment thereof.

3. The protein of claim 2 having an amino acid sequence as shown in SEQ. ID. NO. 1 containing deletions or substitutions of amino acids.

4. The protein of claim 2, wherein the protein is a fragment comprised of the amino acid sequence of SEQ. ID. NO. 1 that lacks an N-terminal sequence, suitably amino acids no 1-19 inclusive in the sequence of SEQ. ID. NO. 1.

5. The protein of claim 3, which has an amino acid sequence corresponding to a portion of the sequence as shown in SEQ. ID. NO. 1 wherein an amino acid sequence binding to a collagen-binding domain of fibronectin (Fn) and comprising the sequence consisting of amino acids (SEQ. ID. NO. 3) QGERGEAGPP, is deleted.

6. The protein of claim 1, wherein the protein has an amino acid composition of 53 glycine residues, 39 serine residues and 38 proline residues evenly distributed in the protein and optionally 13 tyrosine residues in the C-terminal part of the protein.

7. A DNA fragment comprising a nucleotide sequence coding for a protein according to claim 1.

8. The DNA fragment of claim 7, wherein said fragment has a nucleotide sequence as shown in SEQ, ID. NO. 2 or an equivalent thereof.

9. A recombinant DNA molecule comprising a replicable vector, which suitably is an expression vector, and a DNA fragment according to claim 7 or 8 inserted therein.

10. A host cell comprising a DNA fragment in accordance with claim 7.

11. The host cell of claim 10, wherein said cell is a prokaryotic host cell, suitably a prokaryotic host cell comprised of a strain of E. coli.

12. A method of producing the protein of claim 1, or fragments or analogs thereof comprising culturing a host cell as defined in claim 10, and isolating the expression product comprising the protein from the culture.

13. The method of claim 12, wherein said method further comprises purification of the expression product, such as by affinity chromatography.

14. The method of claim 12, which method comprises
(a) introducing the DNA fragment encoding the protein or fragment or analog thereof into an expression vector;
(b) introducing the said vector, which contains the said DNA fragment, into a compatible host cell;
(c) culturing the host cell provided in step (b) under conditions required for expression of the product encoded by said DNA fragment; and
(d) isolating the expressed product from the cultured host cell, and, optionally,
(e) purifying the isolated product from step (d) by affinity chromatography or other chromatographic methods known in the art.

15. A vaccine comprising the fibronectin-binding protein or a fragment or an analog thereof as defined in any one of claims 1-6 or as produced by a method as defined in any one of claims 12-14.

16. The vaccine of claim 15, which vaccine is a vaccine that protects horses against strangles caused by S. equi infection.

17. An antibody specific for a fibronectin-binding protein of any one of claims 1-6 or a fragment or an analog thereof, which antibody is polyclonal or monoclonal, or a fragment of said antibody.

18. An antigenic preparation comprising an antigen consisting of the protein of any one of claims 1-6 or a fragment or an analog thereof.

19. An antiserum comprising an antibody of claim 17, which is comprised of a polyclonal antibody.

20. A method for the production of an antiserum, said method comprising administering an antigenic preparation of claim 18 to an animal host to produce antibodies in said animal host and recovering antiserum containing said antibodies produced in said host animal.

21. A method of prophylactic or therapeutic treatment of S. equi infection in mammals, suitably horses, comprising administering an immunologically effective amount of a vaccine of claim 15 or 16, an antibody of claim 17, or an antiserum of claim 19.


SEQUENCE LISTING (18 sequences)

SEQ ID NO: 1 - protein, 281 aa, Streptococcus equi
MRKTEGRFRTWKSKKQWLFAGAVVTSLLLGAALVFGGLLGSLGGSSHQARPKEQPVSSIGDDDKSHKSSSDQPTNHQHQATSPSQPTAKSSGHHGNQPQSLSVNSQGNSSGQASEPQAIPNQHHQPQGKPQHLDLGKDNSSPQPQPKPQGNSPKLPEKGLNGENQKEPEQGERGLPGLNGENQKEPEQGERGEAGPPSTPNLEGNNRKNPLKGLDGENKPKEDLDGYNHGRRDGYRVGYEDGYGGKKHKGDYPKRFDESSPKEYNDYSQGYNDNYGNGNPD

SEQ ID NO: 2 - DNA, 846 bp, Streptococcus equi
atgagaaaaacagaaggacgttttcgcacatggaagtccaaaaaacaatggctatttgccggtgcagtgggagctgcacttgtctttggaggtttattaggaagtcttggtggctcatcccagcagccagtcagctcgattggagatgacgataagtcgcacaagagctcatcaccaccgaaaaaggataacttgcagcctaagccttcagatcagcctactaatcgcccgtcccagccgacagcaaagagctcaggtcatcatgggaatcaacctcaccaaggaaatagtagtggacaggcctcagagcctcaggctattcctaatcaagggctgagaggaggtaacagctctggttcaggtcatcaccatcagccacaagatctaggtaaggataattctagcccgcagcctcaaccaaagcctcagggcaaaaaaggcttgaatggtgaaaatcagaaggaaccggagcaaggtgaacgaggttcagggttgagtggtaataatcaaggccgtccttcgcttccaggcttgaatgcagagcaaggtgaacgaggtgaagccggtcccccatcaactccgaatttagatcctttaaaaggattagatggagagaataagccaaaggaagatttagacggtaatgatgaatcaccaaaacttaaagacgaacacccctacaatcatggtcgtcgctatgaggatggatatggtggcaaaaagcacaaaggagattatcctaagcgaaaggaatataatgactatagtcaagggtataatgataattatggaaatggcgatagaggtggtaagagaggatacggctattcttacaatcccgactaa

SEQ ID NO: 3 - protein, 10 aa, Streptococcus equi
QGERGEAGPP

SEQ ID NO: 4 - DNA, 32 bp, Artificial Sequence
  Derived from Streptococcus equi subspecies zooepidemicus
cgggatccctattacacattctcatctcatat

SEQ ID NO: 5 - DNA, 27 bp, Artificial Sequence
  Derived from Streptococcus equi subspecies zooepidemicus
ggaattccagaaagcccgcctgtaaac

SEQ ID NO: 6 - DNA, 30 bp, Artificial Sequence
  Derived from Streptococcus equi subspecies equi Bd 3221
acaagccatggagcacttgtctttggaggt

SEQ ID NO: 7 - DNA, 21 bp, Artificial Sequence
  Derived from Streptococcus equi subspecies equi Bd 3221
gtcgggattgtaagaatagcc

SEQ ID NO: 8 - protein, 9 aa, Streptococcus pyogenes
QGERGETGP

SEQ ID NO: 9 - protein, 6 aa, Streptococcus pyogenes
  UNSURE (3)..(3): Xaa equals any amino acid, unknown, or other
LPXTGX

SEQ ID NO: 10 - protein, 10 aa, Streptococcus equi
QGERGEAGPP

SEQ ID NO: 11 - protein, 10 aa, Streptococcus pyogenes
QGERGETGPA

SEQ ID NO: 12 - protein, 10 aa, unknown
  Collagen alpha2 (I) 711
PGERGEVGPA

SEQ ID NO: 13 - protein, 10 aa, unknown
  Collagen alpha1 (I) 991
SGERGPPGPM

SEQ ID NO: 14 - protein, 10 aa, unknown
  Collagen alpha1 (II) 329
PGERGRTGPA

SEQ ID NO: 15 - protein, 10 aa, unknown
  Collagen alpha1 (III) 797
PGERGETGPP

SEQ ID NO: 16 - protein, 10 aa, unknown
  Collagen alpha1 (IV) 319
QGEKGEAGPP

SEQ ID NO: 17 - DNA, 2127 bp, Streptococcus equi
  CDS (108)..(1901)
aaagaaatcagcttaaaactattacacattctcatctcatatgatatagttattcaggtttcctaacggaaaacatattaaaaacttaatatacggagagaagagagttgaaaacaaaatcatttagaaaggtactgacgacgtcagctacctgtattgtgctggcaacaagctttgccggaggaaccctacgcgtctgggcagagcagctttattatgggtggaatgatggaacgagacaaagttcgccatattttttgtacgtatcgcctaaaaatgctccaaagcgtgaattaaaagacgagtatgttgtttattgctttaacaaaaaattgtattggccagatcaatgggaatctatatacagcaattttaatgacatcagatctccatataacgatttacctgtatatgagaaaaaactaggatatgatggtatatttaaacaatatgctccagattacaaaaaagatattagtgatattgcaagtgctttggtggcagttttaagtaatggataccccactaacaagtcacaactatcaactagctaccatttaaataatgattcttctagaaaagttactcaattagccatttggtattttagtgatagtttaacaaaagaataccttaaagatacgggtggttataacttaaacgatatggaaaaaaaagctttagattttttaatcagtaaaggagaggattctaagcttaaatcagagcagagtaattactcattggatatttatgtttatcaaagtggcgggcatgaccatatgaaagattaccaaaatcttctcggctctaccttaattcctaaagaaccgctaaagcctcagctaggtggttttagtggacataatggaaatggattaagcggccttgaaggaggatcatcaggttcacaagaaactaatgaagatggtaagaaaggacttataggtttccatggaggactctcaggaagcgagggcaaacgagatcctttgccaggattgaagggtgaggctggtgcacctgatacacctcaaaagcctaatgatccattgcaaggtcttgaaggcggtaactctcctatagtagaacaaaactatggtagtaccgaaggatatcatggtcaatcaggcattcttgaggaaaccgaagatactaacccacctggtatcatactaggcggctcaggaaatgtcgaaacgcatgaagatactagaaaccctcatctgatggggatcggcggcggtctagctggcgaatcaggagaaacgacacctaaaccaggacaaaccggcgggcaaggaccagtcatcgagacaacagaggatacacaaaaaggcatgtctggacaatctggtggcactatcgagtcagaaaacaccaaaaagccggaggtcatgattggtggtcagggacaaaccatcgagacaacagaggacacacaaaaaggcatgtctggacaatctggcggtactatcgagtcagaggacactaagaaacctgaggtcatgattggtggtcagggacaaatcatcgacttctctgaaaacacccaatcaggtatgtctgggcagtctggtgacactacggtaattgaggataccaagaagtctgagataatcattggtgggcaaggacaaatcatcgacttctctgaggatactcagccgggtatgtctggtcaatctggaggcactacaattgtcgaagacaccaagaagccgacacctaagcctaaacctgcacctgcgccaattgttaatgacgaaaaacctaacaaaggcactcatctcccacagacaagtgatatgaagcaactcaccctaagcatcatcggtgcaatgtcaatgctgcttgtcctatgtctgtctctattcaagcgaccatctaaaaaagactaagcaacactgacaagtgatcattattgtacggacgtttacaggcgggctttctgccctcttaaaacaccgtacaagagcaaaacggtctaaatagaaatatccctcaagggttagacaatcgctctagcccttgggggctttttttgacatttataatagaagagagcaagcagtgacactttctttttgccctatatctgttaacactagcggtatgtgcagttac

SEQ ID NO: 18 - protein, 597 aa, Streptococcus equi
LKTKSFRKVLTTSATCIVLATSFAGGTLRVWAEQLYYGWNDGTRQSSPYFLYVSPKNAPKRELKDEYVVYCFNKKLYWPDQWESIYSNFNDIRSPYNDLPVYEKKLGYDGIFKQYAPDYKKDISDIASALVAVLSNGYPTNKSQLSTSYHLNNDSSRKVTQLAIWYFSDSLTKEYLKDTGGYNLNDMEKKALDFLISKGEDSKLKSEQSNYSLDIYVYQSGGHDHMKDYQNLLGSTLIPKEPLKPQLGGFSGHNGNGLSGLEGGSSGSQETNEDGKKGLIGFHGGLSGSEGKRDPLPGLKGEAGAPDTPQKPNDPLQGLEGGNSPIVEQNYGSTEGYHGQSGILEETEDTNPPGIILGGSGNVETHEDTRNPHLMGIGGGLAGESGETTPKPGQTGGQGPVIETTEDTQKGMSGQSGGTIESENTKKPEVMIGGQGQTIETTEDTQKGMSGQSGGTIESEDTKKPEVMIGGQGQIIDFSENTQSGMSGQSGDTTVIEDTKKSEIIIGGQGQIIDFSEDTQPGMSGQSGGTTIVEDTKKPTPKPKPAPAPIVNDEKPNKGTHLPQTSDMKQLTLSIIGAMSMLLVLCLSLFKRPSKKD